(19) United States
(12) Patent Application Publication
(10) Pub. No.: US 2020/0155807 A1
(43) Pub. Date: May 21, 2020

(54) SMALL DIAMETER CATHETER FOR INTRODUCTION INTO THE TRACHEA AND OTHER ORIFICES, AS WELL AS INTO PASSAGES THAT ARE DIFFICULT TO INTUBATE OR ACCESS

(71) Applicant: The Board of Trustees of the Leland Stanford Junior University, Palo Alto, CA (US)

(72) Inventors: Ronald S. Cohen, Palo Alto, CA (US); Juan N. Walterspiel, Menlo Park, CA (US)

(73) Assignee: The Board of Trustees of the Leland Stanford Junior University, Palo Alto, CA (US)

(21) Appl. No.: 16/434,158

(22) Filed: Jun. 6, 2019

Related U.S. Application Data
(60) Provisional application No. 62/834,933, filed on Apr. 16, 2019.
(60) Provisional application No. 62/681,727, filed on Jun. 7, 2018.

(51) Int. Cl. A61M 25/04; A61M 27/00; A61M 25/00; A61B 10/02; A61M 16/04

(52) U.S. Cl. CPC A61M 25/04; A61M 27/00; A61M 25/0026; A61M 25/0054; A61M 25/0041; A61M 25/003; A61M 2240/00; A61M 16/0486; A61M 2210/1085; A61M 2210/101; A61M 2210/122; A61M 2210/1017; A61B 10/02

(57) ABSTRACT

A catheter includes: a tubular member having a first end, a second end, a body extending between the first end and the second end, and a first lumen in the body; a first flexible line having at least a segment located in the first lumen of the body; and a first anchor at an end of the first flexible line, the first anchor having a cross sectional dimension that is larger than a cross sectional dimension of the first lumen; wherein the first anchor is configured to apply a compression force against an exterior surface at the first end of the tubular member in response to tension applied through the first flexible line.

### RELATED APPLICATION DATA

This application claims priority to, and the benefit of, U.S. Provisional Patent Application No. 62/834,933 filed on Apr. 16, 2019, and U.S. Provisional Patent Application No. 62/681,727 filed on Jun. 7, 2018. The entire disclosures of the above applications are expressly incorporated by reference herein.

### FIELD

This disclosure relates generally to catheter systems and methods to intubate and to aspirate or instill therapeutic formulations or diagnostic specimens into or from the trachea as well as other orifices and passages that are difficult to intubate or access. In some embodiments, features described herein may be implemented as additions and/or modifications to existing pliable, small diameter single or multi-lumen catheters, which allow such catheters to be converted into temporarily less pliable and curved configurations in order to facilitate their insertion into orifices and body conduits that are difficult to catheterize. Some of the embodiments specifically relate to systems and methods for the tracheal intubation of premature neonates and also adults to instill surfactant. Further embodiments relate to bending of the catheter tips into pliable hook or pig tail configurations in order secure such catheters in the bladder, the pericardium, the peritoneal or pleural space, and any other cavity that requires irrigation and drainage. These catheters and the ends of their traction lines may also be modified to collect one or more uncontaminated biological specimens from areas that are otherwise difficult to reach.

### BACKGROUND

Approximately four million infants are born each year in the United States to a population of approximately 330 million. About 10% of all infants are delivered prematurely and approximately 1% of all neonates exhibit a respiratory distress syndrome that is common in infants of less than 30 weeks of gestation and under 1,200 gm. of birth weight. This respiratory distress syndrome is caused by lung immaturity and insufficient generation of surfactant.

Worldwide about 72 million infants each year require surfactant administration and endotracheal intubation. However some of the neonates can be treated successfully with less invasive respiratory assistance which is administered by continuous positive airway pressure (CPAP), initially applied to an infant's airways through a nose or face mask. With CPAP, and in absence of an endotracheal tube, the surfactant has to be instilled into the trachea through a small catheter. This is difficult to accomplish and is currently done through a small umbilical catheter or a thin feeding tube, which range in size from 3.0 to 9.0 French. [The French (Fr) number corresponds to three times the outer diameter of a catheter measured in millimeters].

The above referred to tubes and catheters have the disadvantage of not being made for this purpose and they are very soft and pliable, which makes it exceedingly difficult to maneuver them over the small epiglottis into the trachea of a premature neonate.

This hinders the above less invasive technique for administering surfactant from being used routinely and leads to otherwise unnecessary intubations with their sometime detrimental long term consequences.

Some embodiments described herein allow for the proposed catheters to be retained in the bladder, the pericardium, the peritoneal or pleural space, and any other cavity that requires irrigation and drainage by reversibly bending the catheter's end into a pliable hook or pig tail configuration.

Further embodiments of the end of the traction lines within the proposed catheter according to this disclosure allow for the collection of uncontaminated diagnostic specimens from areas that are otherwise difficult to reach.

### SUMMARY

A solution for difficult intubations is disclosed that stiffens and curves thin catheters for this purpose. The end of the catheter(s), according to embodiments of this disclosure, can be coiled into reversible pig tail or hook configurations for retention within the bladder, the pericardial space, the pleural space, the peritoneal space, abscesses, and other spaces that need to be irrigated or drained.

The proposed catheters containing the lines can be used for the sampling of uncontaminated specimens from areas that are otherwise difficult to reach.

In general, any single or multi-lumen thin, soft and all too pliable catheter is stiffened from within with a fitting thin line in one or more of its lumina, The fitting line is made of nylon®, polyvinylidene, ultra-high-molecular-weight polyethylene, Dacron® or other suitable material of sufficient diameter, length, and tensile strength known to those skilled in this art.

Such a line(s) that is/are introduced into the lumen of a single or multi-lumen catheter(s) exit(s) at the distal end of the catheter, or in embodiments according to this proposal, where a catheter has more than two lumina and lines, these from hereon called traction lines, exit at additional suitable distance(s) proximal to the catheter's end.

The tips of the line(s) are affixed to or contiguous (i.e. consist of molted line material) to a thickened ball- or football shaped structure that in some embodiments can have perforations to allow flow from and into the catheter.

The distal thickenings of the line(s) are larger in diameter than the lumen housing the line(s) to prevent the line(s) from being pulled into their respective lumina, and in some embodiments smooth enough to seal their lumina during the catheter's introduction.

Traction on the line(s) stiffens the thin and floppy single or multi-lumen catheter(s), and curves them for easy introduction, and in some embodiments as described above into hook or pigtail configurations for a-traumatic retention.

In some embodiments, one or more of the catheters described herein allow for distal end of the catheter to, under traction, form pigtail configuration or otherwise diagonal footprint with enlarged configuration that would retain the catheter relative to the patient. The atraumatically secured catheter may then be removed from the patient atraumatically, after releasing the traction with the catheter's end straightening out.

In some embodiments selected catheter segments can be curved by tensing additional lines in a multi-lumen catheter with those additional tension lines exiting and being engaged proximally from a catheter's tip and proximal to the catheter segments to be curved.

In other embodiments portions at the tip of the catheter are thinned on one side in a linear or in a spiral-like fashion, so that the additional tension on a line results in the formation of a reversible relatively soft hook or pig tail configuration of to secure and retain a catheter in the bladder, the pericardium, the pleural or peritoneal space, or any other cavity that requires irrigation and drainage.

All lines exit through suitable proximal ports for each lined lumen of a catheter and enter into a continuously affixed or connected device of a known design, that allow for the tensing of a line and maintaining it under tension as needed.

In one embodiment, the device suggested is a one way stopcock, where a the rotation of the valve entraps and arrests a line, while also closing the respective conduit to the outside so that nothing can enter into or escape from that conduit.

In a further embodiment of this proposal the distal part of a line is configured to seal its respective lumen upon traction and the distal segment has small perforations, dimples, riffles, mini hooks, bristles, loops, layered and non layered absorbents, a suitable arrangement of hydrophobic and hydrophilic surfaces that are known to and can be selected by those skilled in the art in order to collect biological samples. These samples would be relatively uncontaminated and can be collected from areas that are otherwise very difficult to reach.

These uncontaminated samples are collected by first extruding and then retracting line(s) followed by removal of a catheter.

One or more embodiments of the catheters according to this proposal can serve as a lead over which larger catheters can be slid in order to reach their destinations.

In some embodiments, a catheter or, in general, a pliable tube, is disclosed herein, wherein the tube may be stiffened internally with an elongated structure, such as a thin nylon, polyvinylidene, ultra-high-molecular-weight polyethylene, Dacron®, or any of other lines made from different materials. Such a line may loop out from a distal exit port of the tube, where the loop will be in plane with the vocal cords aided by a slanted distal tip surface (e.g., a V or U shaped incision on the concave side) of the tube. This relatively soft loop has the function of providing the tube with a blunt advancing end that facilitates the tip's introduction into a human body (e.g., into the trachea and advancement to below the vocal cords, or into other body site(s)). The line forming the loop may go back into the tube. The part of the line that bends into the loop may have a larger cross sectional dimension (e.g., diameter) than the rest. The two parallel lines exit the proximal end of the tube through a fluid tight split membrane exit port, or other opening of similar function to seal and arrest the lines. The proximal end of the tube is Y shaped, allowing for two separate entry/exit channels that merge towards the distal (in or towards the patient) end of the tube. One of these arms of the Y-portion that ends proximally of the tube, end in a port through which the lines exit. Either one or both of the lines may be pulled to adjust the size of the loop at the distal end of the catheter, and/or to adjust a tube stiffness and curvature to facilitate intubation. The other one of the arms of the Y-portion is the entry port for the injection or aspiration of a substance (e.g., the surfactant or other preparation or biological fluid). In some embodiments, the insertion of the catheter into the patient may be done by visualizing the epiglottis and taking advantage of the curvature of the tube, and by sliding the loop or otherwise blunted tip over the epiglottis into the trachea, thereby avoiding entry into the esophagus. In some embodiments, the blunted tip of the catheter may be oriented so that the plane of the tip is in plane of the vocal cords. In lieu of forming a loop, the flexible line may also be threaded through, or attached to a suitably sized bead outside of the tip of the catheter or tube, thus rendering the similar function of blunting the advancing end of the tip of the tube and preventing the line(s) from being pulled into the tube, while the tube's optimal pliability and curvature is adjusted by traction on the exiting line(s). Such a ball or bead may have perforation(s) to facilitate the passage of the injected or aspirated surfactant or other preparation or biological fluid. In other embodiments, the catheter may include a single line forming the distal loop or attaching to the above mentioned ball or bead. In some embodiments, the tube or catheter may optionally have a thickened cross section (e.g., by thickening the wall or by adding a shaping element to the wall) that extends continuously or intermittently longitudinally along the tube. The thickened cross section may be implemented on the inner curvature (i.e., the side of the tube that undergoes compression during bending) and/or may be implemented on the exterior curvature (i.e., the side of the tube that undergoes tension during bending). The thickened cross section provides the tube or catheter with the desired stiffness and curvature. The choice of these alternative embodiments may be influenced by the anatomical dimensions of the intended application(s), the materials for the tube(s) and the line(s), and the corresponding considerations for economic production and the size of the market to be served or created.

A catheter includes: a tubular member having a first end, a second end, a body extending between the first end and the second end, and a first lumen in the body; a first flexible line having at least a segment located in the first lumen of the body; and a first anchor at an end of the first flexible line, the first anchor having a cross sectional dimension that is larger than a cross sectional dimension of the first lumen; wherein the first anchor is configured to apply a compression force against an exterior surface at the first end of the tubular member in response to tension applied through the first flexible line.

Optionally, in a first operational mode, the first anchor is configured to engage against the first end of the tubular member to allow a bending of the tubular member to occur based on a tension applied through the first flexible line; and wherein, in a second operational mode, the first anchor is moveable away from the first end of the tubular member in response to a pushing force applied through the first flexible line.

Optionally, the tubular member comprises a second lumen in the body.

Optionally, the tubular member comprises a third lumen in the body.

Optionally, the first anchor comprises a spherical configuration or an ellipsoidal configuration.

Optionally, the first anchor and the first flexible line are made from different respective materials.

Optionally, the first anchor and the first flexible line are made from a same material.

Optionally, the first anchor and the first flexible line have an unity configuration.

Optionally, the first anchor and the first flexible line are integrally formed together, and wherein the first anchor is a thickened tip of the first flexible line.

Optionally, in the first operational mode, the tubular member is configured to form a pig-tail configuration in response to the tension applied through the first flexible line.

Optionally, in the first operational mode, the tubular member is configured to form a reversible catheter-retaining hook in response to the tension applied through the first flexible line.

Optionally, the tubular member has a port at the first end of the tubular member, and wherein the first anchor is configured to close the port when in the first operational mode.

Optionally, the first anchor is configured to fluidly seal the port when in the first operational mode.

Optionally, the first anchor and/or at least a part of the first flexible line comprises a sampler.

Optionally, the sampler is configured to obtain a biological sample.

Optionally, the sampler comprises bumps and/or holes.

Optionally, the sampler comprises an absorbent.

Optionally, the absorbent is perforated.

Optionally, the sampler comprises hydrophilic loops and/or hooks.

Optionally, the sampler comprises hydrophobic loops and/or hooks.

Optionally, the catheter further includes a second flexible line.

Optionally, the end of the first flexible line and an end of the second flexible line are configured to couple to the tubular member at different respective longitudinal positions along a longitudinal axis of the tubular member.

Optionally, the end of the first flexible line and an end of the second flexible line are configured to couple to the tubular member a same longitudinal positions along a longitudinal axis of the tubular member.

Optionally, the catheter further includes a second anchor at an end of the second flexible line.

Optionally, the first anchor comprises a loop formed by an elongated element.

Optionally, the elongated element is an extension of the first flexible line.

Optionally, a first end of the loop extends to the first flexible line, and a second end of the loop extends to a second flexible line, and wherein a size of the loop is adjustable via manipulation of the first flexible line and/or the second flexible line.

Optionally, the loop has a bending limit that prevents the loop from being completely pulled inside the tubular member.

Optionally, the first anchor comprises a sphere or ellipsoid with a fluid delivery channel.

Optionally, the first flexible line comprises a fluid delivery lumen.

Optionally, the first end of the tubular member has a distal port on a distal tip surface, the distal tip surface forming an acute angle with respect to a longitudinal axis of the tubular member.

Optionally, the first end is configured to align a plane of the first anchor with a vocal cord of a patient.

Optionally, the catheter further includes a shaping element located at the first end of the tubular member, wherein the shaping element is coupled to a wall of the tubular member, and is configured to bend the first end of the tubular member.

Optionally, the shaping element is configured to apply tension or compression on one side of the tubular member.

Optionally, the catheter further includes a first port at the second end configured to accommodate the first flexible line.

Optionally, the catheter further includes a second port at the second end configured to allow fluid to be delivered therethrough.

Optionally, the catheter further includes a source of treatment fluid coupled to the second port.

A medical method includes: inserting a part of a tubular member into a patient, the tubular member having a first end, a second end, a body extending between the first end and the second end, and a first lumen in the body; and bending the tubular member using a first flexible line and a first anchor at an end of the first flexible line, wherein at least a segment of the first flexible line is located in the first lumen of the body, the first anchor having a cross sectional dimension that is larger than a cross sectional dimension of the first lumen, wherein the first anchor is configured to apply a compression force against an exterior surface at the first end of the tubular member in response to tension applied through the first flexible line.

Optionally, the method further includes moving the first anchor away from the first end of the tubular member by applying a pushing force through the first flexible line to expose a part of the first flexible line outside the tubular member.

Optionally, the tubular member is bent to form a pig-tail configuration in response to the tension applied through the first flexible line.

Optionally, the tubular member is bent to form a reversible catheter-retaining hook in response to the tension applied through the first flexible line.

Optionally, the tubular member has a port at the first end of the tubular member, and wherein the method further comprises closing the port using the first anchor.

Optionally, the method further includes collecting a biological sample using a sampler at the first anchor and/or at a part of the first flexible line.

Optionally, the method further includes delivering a substance to a location inside the patient using the first lumen or another lumen in the tubular member.

Optionally, the method further includes advancing the first end of the tubular member over an epiglottis of a patient, and advancing the first end of the tubular member into a trachea of the patient.

A medical method includes: inserting a part of a tubular member into a patient, the tubular member having a first end, a second end, a body extending between the first end and the second end, and a first lumen in the body; bending the tubular member using a first flexible line and a first anchor at an end of the first flexible line, wherein at least a segment of the first flexible line is located in the first lumen of the body, the first anchor having a cross sectional dimension that is larger than a cross sectional dimension of the first lumen, wherein the first anchor is configured to engage against the first end of the tubular member; and moving the first anchor away from the first end of the tubular member by applying a pushing force through the first flexible line to expose a part of the first flexible line outside the tubular member.

Optionally, the tubular member is bent to form a pig-tail configuration in response to tension applied through the first flexible line.

Optionally, the tubular member is bent to form a reversible catheter-retaining hook in response to tension applied through the first flexible line.

Optionally, the tubular member has a port at the first end of the tubular member, and wherein the method further comprises closing the port using the first anchor.

Optionally, the act of closing the port comprises using the first anchor to fluidly seal the port.

Optionally, the method further includes collecting a biological sample using a sampler at the first anchor and/or at the part of the first flexible line.

Optionally, the method further includes delivering a substance to a location inside the patient using the first lumen or another lumen in the tubular member.

Optionally, the tubular member is also bent using a second flexible line and a second anchor at an end of the second flexible line.

Other and further aspects and features will be evident from reading the following detailed description of the embodiments.

### BRIEF DESCRIPTION OF THE DRAWINGS

The drawings illustrate the design and utility of embodiments, in which similar elements are referred to by common reference numerals. In order to better appreciate how the above-recited and other advantages and objects are obtained, a more particular description of the embodiments will be rendered, which are illustrated in the accompanying drawings. These drawings depict only typical embodiments and are not therefore to be considered limiting of its scope.

FIG. 1 is a schematic drawing of a dual lumen catheter with one of the lumina filled with a traction line. Traction applied on that line results in stiffening and curving of the catheter.

FIG. 2 is a schematic drawing of a triple lumen catheter with a first traction line exiting from an end of the catheter, and a second traction line exiting proximally from to the end of the catheter, which allows bending of the catheter independently at two locations and/or collection of samples from more than one sites at different locations along the catheter.

FIG. 3A is a schematic drawing of a part of a catheter that is reversibly formed into a pig tail configuration.

FIG. 3B is a schematic drawing of a part of a catheter that is reversibly formed into a securing hook.

FIG. 4 is a schematic drawing of a catheter's traction line whose distal portion is configured to facilitate sampling of uncontaminated biological specimens from sites that otherwise would be difficult to reach.

FIG. 5 illustrates a catheter with a loop tip at a distal end of the catheter and a tube attached to the loop.

FIG. 6 illustrates a catheter with a tip-blunting bead having a channel, and a tube attached to the bead.

FIG. 7A illustrates a catheter with a loop and a single line attached or extending to the loop.

FIG. 7B illustrates a catheter with a loop and a single line attached or extending to the loop.

FIG. 7C illustrates a catheter with a loop.

FIG. 8 illustrates a catheter with a tip-blunting bead having a channel, and a single line attached to the bead.

FIG. 9 illustrates a cross section of a catheter with a bending element configured to apply a tension on one side of the catheter to bend the catheter.

FIG. 10 illustrates a cross section of a catheter with a bending element configured to apply a compression on one side of the catheter to bend the catheter.

FIG. 11 illustrates a distal end of a catheter in accordance with some embodiments.

FIG. 12 illustrates a placement of a catheter in accordance with some embodiments.

### DESCRIPTION OF THE EMBODIMENTS

Various embodiments are described hereinafter with reference to the figures. It should be noted that the figures are not drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated.

This disclosure relates generally to catheter systems and methods to intubate orifices of the human or an animal's anatomy in order to deliver or extract liquids or substance, and more specifically, to introduce a stiffened, blunted catheter into a trachea and beyond the vocal cords of a premature neonate. These catheters may also be used in adults. It is disclosed herein design of thin and pliable catheters that facilitate their introduction into any orifices and body cavities that are difficult to intubate. These include, but are not limited to bronchi, sinuses, billiary and pancreatic duct, intestine, urethra, bladder, ureters, calices, vagina, uterus and anexa, as well as any other naturally occurring or created tracts.

Once in place, such catheters can serve as leads for larger catheters to be slid over. Alternatively, or additionally, such catheters may be used to provide treatment (such as delivery of medical substances and/or drainage), and/or to obtain biopsy.

In further embodiments the catheters according to this disclosure have more than one lumen filled with traction lines and those can exit at various locations proximal to the catheter's end. This arrangement allows for the catheters to be bent at different locations according to the site specific needs for proper navigation and advancement.

In some embodiments, a distal part of the catheter may have longitudinal or suitable partially circumferential thinnings or enforcements, so that upon traction on a traction line, the end of the catheter will coil into a reversible hook, pig tail, or any of other configurations, to retain the catheter in the bladder, pericardial, pleural or peritoneal space, and in any other cavity that needs to be drained and irrigated.

In further embodiments, traction line(s) in catheters according to this disclosure may have additionally treated, configured, or specifically covered surfaces that allow for the adherence and absorption of biological specimens for diagnostic purposes. Catheters with such traction line(s) may be used to collect relatively uncontaminated biological specimens from areas that are otherwise difficult to reach.

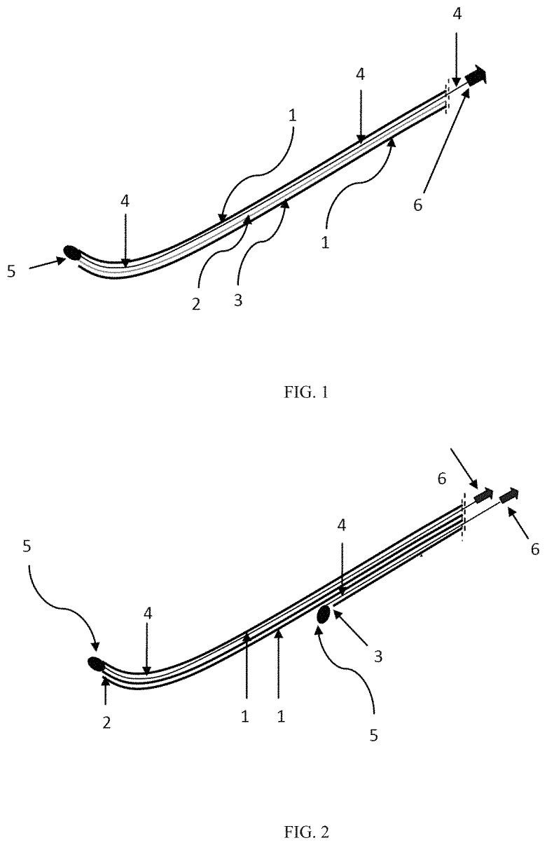

FIG. 1 illustrates a catheter in accordance with some embodiments. The catheter includes a tubular member 1 having a first end, a second end, a body extending between the first end and the second end. The catheter also includes a first lumen 2 in the body, a first flexible line 4 having at least a segment located in the first lumen 2 of the body, and a first anchor 5 at an end of the first flexible line 4. The first anchor 5 has a cross sectional dimension that is larger than a cross sectional dimension of the first lumen 2. The first anchor 5 is configured to apply a compression force against an exterior surface at the first end of the tubular member 1 in response to tension applied through the first flexible line 4.

The first flexible line 4 may be made from nylon, polyvinylidene, ultra-high-molecular-weight polyethylene, Dacron®, or other suitable synthetic or natural material.

In some embodiments, in a first operational mode, the first anchor 5 is configured to engage against the first end of the tubular member 1 to allow a bending of the tubular member 1 to occur based on a tension applied through the first flexible line 4. In a second operational mode, the first anchor 5 is moveable away from the first end of the tubular member 1 in response to a pushing force applied through the first flexible line 4.

In the illustrated embodiments, the tubular member 1 also has a second lumen 3 in the body. The second lumen 3 may be used to deliver a substance into a body of a patient (e.g., delivery of surfactant to neonate or an adult), and/or to remove a substance (e.g., for drainage, biopsy, etc.). The second lumen 3 may also be used to deliver another medical device into the patient. In other embodiments, the tubular member 1 does not include the second lumen 3, and the catheter includes only a single lumen 2.

In other embodiments, the catheter may include more than two lumens (e.g., three lumens, four lumens, etc.).

During use, the catheter is inserted into a body of a patient through a natural opening or a man-made opening. The flexible line 4 may be pulled at the proximal end of the catheter to apply tension at the flexible line 4. The flexible line 4 functions as a traction line, which stiffens the tubular member 1 and bends the tubular member 1. The bending of the tubular member 1 steers the distal end (the first end) of the tubular member 1, thereby allowing the catheter to be navigated through bends in the human body. In the illustrated embodiments, the first anchor 5 is thicken so that it has a cross sectional dimension larger than the first lumen 2 to prevent the first anchor 5 from being pulled into the first lumen 2. After the catheter 1 has been desirably placed inside the patient, the flexible line 4 may be relaxed, and may be pushed distally to move the anchor 5 distally with respect to the distal end of the catheter, thereby opening the port at the distal end of the first lumen 2. Substance, such as medication (e.g., surfactant), may then be delivered from the distal end of the catheter to the distal end via the first lumen 2, and exits from the port at the distal end of the first lumen 2. Alternatively, if the catheter includes a second lumen 2, the anchor 5 may not need to be advanced distally, and may remain coupled to the distal end of the catheter. In such cases, the second lumen 2 may be used to deliver substance to the patient. In some cases, the first lumen 2 or the second lumen 2 may be used to collect substance from inside the patient. The substance may be biological fluid that is desired to be drained from the patient. Alternatively, the substance may be tissue that is desired to be collected as biological sample.

In some embodiments, the anchor 5 may be configured to form a seal to cover the port at the distal end of the lumen 2. In such cases, when the flexible line 4 is tensioned to pull the anchor 5 proximally, the anchor 5 functions as a sealing plug to close the port at the distal end of the lumen 2.

In some embodiments, the catheter may optionally also include a control at the proximal end of the tubular member 1 to regulate and/or maintain the tension of the flexible line 4. For example, such control may include a stopcock (e.g., a one way stopcock, two way stopcock, three way stopcock, etc.). Also, in some embodiments, the control may include a knob, wherein torsion applied to the knob will apply tension to the flexible line 4 and/or will clamp the flexible line 4 going through a valve. Accordingly, in some cases, manipulation of the control may simultaneously apply tension to the flexible line 4 and seal the lumen 2 via the anchor 5.

FIG. 2 illustrates a catheter in accordance with other embodiments. The catheter is the same as that described with reference to FIG. 1, except that the catheter includes a second flexible line 4. In particular, the tubular member 1 includes a second lumen, and the catheter includes a first flexible line 4 in a first lumen 2, and a second flexible line 4 in the second lumen 3 of the tubular member 1. The catheter also includes a second anchor 5 at the distal end of the second flexible line 4. During use, one of the two flexible lines 4 may be selectively pulled at the proximal end of the catheter to bend the distal end of the catheter in a desired direction. In the illustrated embodiments, the anchors 5 are at different longitudinal positions along a longitudinal axis of the catheter. This allows the catheter to be bent at separate longitudinal locations. In other embodiments, the anchors 5 may be at the same longitudinal position with respect to the longitudinal axis of the catheter. Such configuration allows the catheter to be bent towards different directions (e.g., opposite directions (180° apart), 90° apart, etc.).

The catheter may be configured to form different shapes in different embodiments upon tensioning of the flexible line(s) 4.

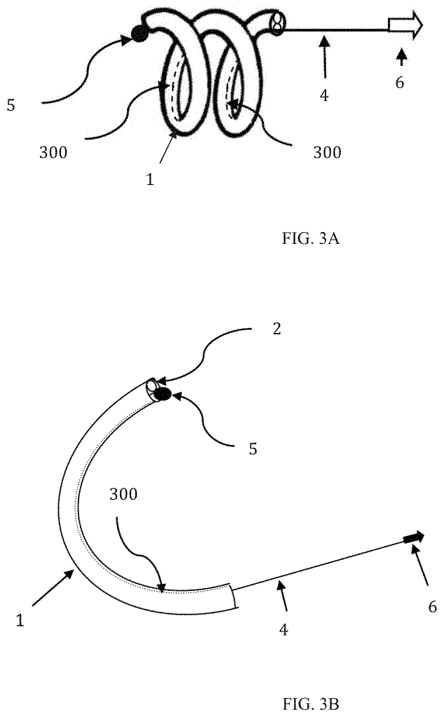

FIG. 3A illustrates an example of a catheter's end that is formed into a reversible pig tail configuration by applying tension to the proximal end of the first flexible line 4. In some embodiments, the wall at the distal end of the tubular member 1 may have a continuous thinning 300 extending longitudinally and in a spiral fashion, or may have individual discrete thinning(s) 300 disposed at different parts of the tubular member 1, that allows the tubular member 1 to coil into the pigtail configuration upon tensioning of the flexible line 4. The thinning(s) 300 may be implemented at the exterior surface or at the interior surface of the wall of the tubular member 1. The pigtail configuration may be utilized to secure the catheter inside the patient. For example, the pigtail configuration may secure the catheter in the bladder, the pericardial, pleural or the peritoneal space, and in any other cavity that needs to be treated, drained, and/or irrigated.

FIG. 3B illustrates an example of a catheter's end that is formed into a reversible hook configuration by applying tension to the proximal end of the first flexible line 4. In some embodiments, the wall at the distal end of the tubular member 1 may have a continuous thinning 300 extending longitudinally, or may have individual discrete thinning(s) 300 disposed at different parts of the tubular member 1, that allows the tubular member 1 to form into the hook configuration upon tensioning of the flexible line 4. The thinning(s) 300 may be implemented at the exterior surface or at the interior surface of the wall of the tubular member 1. The hook configuration may be utilized to secure the catheter inside the patient. For example, the hook configuration may secure the catheter in the bladder, the pericardial, the pleural or peritoneal space, and in any other cavity that needs to be treated, drained, and/or irrigated.

In some embodiments, the catheter described herein may have sampling capability. For example, the flexible line 4 and/or the anchor 5 may be configured to collect samples from inside the patient.

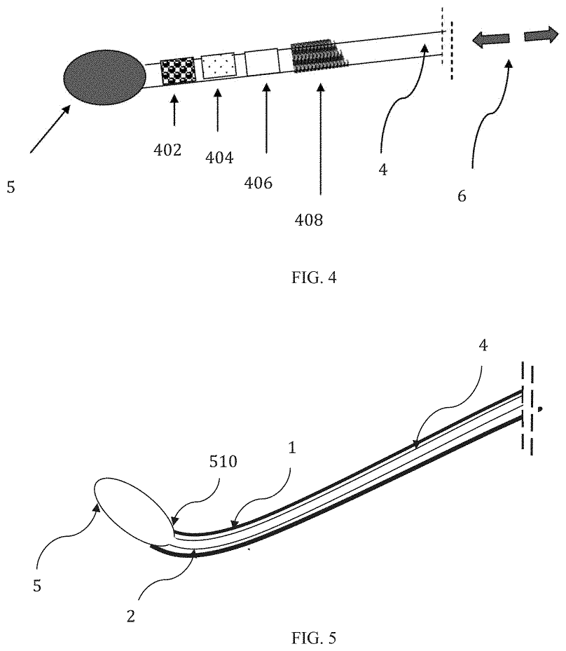

FIG. 4 illustrates a catheter in accordance with other embodiments, particularly showing the catheter having a sampler configured to adhere to or absorb biological samples. The sampler may include bumps and holes 402, perforated absorbent 404, no perforated layered or non-layered absorbent(s) 406, hydrophobic or hydrophilic mini hooks and loops 408, and any combinations thereof. In the illustrated examples, the sampler is implemented on the flexible line 4. In other embodiments, the sampler may be implemented on the anchor 5, or on other structure that may be inserted into the catheter and exits through the distal end of the catheter. In some embodiments, the flexible line(s) 4 may serve the dual function of stiffening and curving the catheter to allow for introduction into difficult to reach areas, to seal their respective lumina and to sample, in a sterile fashion, uncontaminated biological specimens through the catheter by the extrusion and subsequent retraction of their distal tips.

It should be noted that the anchor 5 is not limited to that described in the above embodiments, and that the anchor 5 may have other configurations in other embodiments.

FIG. 5 illustrates a catheter according to other embodiments. The catheter includes a tubular member 1 having a first end, a second end, and a body extending between the first end and the second end. The catheter also includes a lumen 2, a flexible line 4 located in the lumen 2, and an anchor 5 at the distal end of the flexible line 4. In the illustrated embodiments, the anchor 5 is a loop, and the flexible line 4 is a tube with a channel configured to deliver and/or extract substance. During use, the catheter is advanced inside the patient, and the loop protects against abrasion due to its smooth profile. As the catheter is being advanced, the flexible line 4 may be pulled from the proximal end to apply tension at the flexible line 4. The anchor 5 anchors against the distal tip of the tubular member 1 as the flexible line 4 is being pulled, thereby allowing the tubular member 1 to bend into a desired direction and configuration. The bending of the tubular member 1 may allow the catheter to be steered as the catheter is being advanced inside the patient. The bending of the tubular member 1 may also allow the catheter to be anchored against tissue after the catheter has been desirably advanced inside the patient. After the catheter has been desirably placed inside the patient, the channel in the flexible line 4 may deliver a substance (e.g., medication such as surfactant) into the patient. Additionally, or alternatively, the flexible line 4 may be used to extract substance from inside the patient. For example, suction may be applied at the proximal end of the flexible line 4 to extract substance (e.g., biological sample) at the distal end of the flexible line 4. The loop is advantageous because it allows substance to be delivered into the patient, or allows substance to be extracted from the patient, without obstruction of the flow.

In some embodiments, the loop may be made from the same material as that of the flexible line 4. In other embodiments, the loop and the flexible line 4 may be made from different materials. Also, in other embodiments, instead of the flexible line 4 being a tube, the flexible line 4 may be any elongated structure, such as a string, a fiber, a fishing wire, a nylon, etc. In such cases, the loop may be an extension of the elongated structure forming the flexible line 4. For example, one end of the loop may extend from the elongated structure forming the flexible line 4, and the other end of the loop may connects to the distal end of the flexible line 4. Alternatively, the other end of the loop may connects to, or extends from, another flexible line 4 which extends from the distal end of the tubular member 1 to the proximal end of the tubular member 1. In such cases, one or both of the flexible lines 4 may be pulled from the proximal end to adjust the size and shape of the loop. The loop may have a bending limit that prevents the loop from being completely collapsed into the lumen 2 housing one or both of the flexible lines 4. When the loop is collapsed into its smallest size, the loop functions as an anchor so that it anchors against an exterior tip surface of the tubular member 1. This allows the tubular member 1 to be bent in response to tension force applied at one or both of the flexible lines 4.

In one implementation, the first flexible line 4, the loop, and the second flexible line 4, may be implemented using a single elongated structure. For example, the elongated structure may extend along the length of the tubular member 1 to form the first flexible line 4, exits the distal end of the tubular member 1 to form the loop, and then loops back into the tubular member 1 and extends proximally to form the second flexible line 4.

In other embodiments, the tubular member 1 of the catheter of FIG. 5 may have a Y configuration ending in two separate ports. One of ports accommodates the flexible line 4. In particular, the flexible line 4 may exits through one of the ports, which allows for the bending and adjustment of the curvature of the tubular member 1. The other port may be used to deliver substance into the patient, and/or to extract substance from the patient.

In some embodiments, the catheter may be configured (e.g., sized and/or shaped) for insertion into a mouth of a patient, and for advancement over the epiglottis and into the trachea below the vocal cords. For example, the flexible line(s) 4 may be pulled from the proximal end of the catheter to bend the distal end of the catheter at a desired direction and with a desired curvature, to thereby allow passage over the epiglottis and into the trachea. Also, the flexible line(s) 4 may be pulled or pushed to adjust the size and plane of the loop in some embodiments. In addition, in some embodiments, the catheter may be configured to place the plane of the loop at a certain orientation. For example, the distal end of the tubular member 1 of the catheter may have a shape or profile (e.g., a U or V shape orifice facing a direction of bending of the tubular member 1) that biases the loop to orient in a certain direction when the flexible line 4 is pulled to place the loop in abutment with the distal end of the tubular member 1. In some embodiments, the above features may be implemented to place the plane of the loop to be parallel with the vocal cords when the catheter is inserted into a patient. This allows the distal end of the catheter to be easily slided over the epiglottis into the trachea of the patient.

In some embodiments, the junction 510 between the loop and the flexible line 4 may be made more rigid than the flexible line 4 and the loop. This may allow the loop to retain a certain shape and size, without being pulled into the lumen 2. In some embodiments, to stiffen and shape the loop, the part of the line that bends into the loop may have a larger cross sectional dimension than the rest of the line.

In other embodiments, the loop may have a bending limit that prevents the loop from being pulled into the lumen 2. For example, the loop may be made from an elongated structure that resists tight bending. In such cases, the ends of the loop will wedge themselves into the edge of the lumen 2 when the loop is pulled to its limit. In particular, the cross sectional dimension (e.g., diameter) of the elongated structure forming the loop may be properly sized, and the elongated structure may be made to have certain bending stiffness so that when the loop is collapsed to a certain size, the loop will reach the bending limit to prevent the loop from further being collapsed. As a result the ends of the loops will wedge into the edge of the lumen 2. In some embodiments, the loop may have a stopper to prevent the loop from being completely pulled into the lumen 2. For example, the elongated structure forming the loop may be used to tie a knot to form the stopper. Alternatively, a part of the elongated structure may be heated (e.g., by a cigarette lighter, a match, or a commercial heater) to form a blob that functions as a stopper.

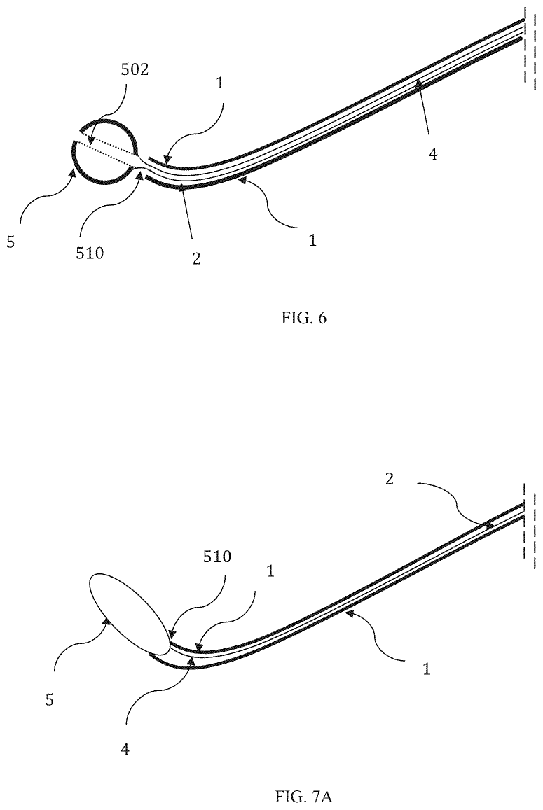

FIG. 6 illustrates a catheter in accordance with other embodiments. The catheter is the same as that in FIG. 5, except that the anchor 5 is a bead. The bead may have a spherical configuration, or an ellipsoid configuration. The bead includes a channel extending therethrough, and is in fluid communication with the channel in the flexible line 4. During use, the catheter is advanced inside the patient, and the bead protects against abrasion due to its smooth profile. As the catheter is being advanced, the flexible line 4 may be pulled from the proximal end to apply tension at the flexible line 4. The anchor 5 anchors against the distal tip of the tubular member 1 as the flexible line 4 is being pulled, thereby allowing the tubular member 1 to bend into a desired direction and configuration. The bending of the tubular member 1 may allow the catheter to be steered as the catheter is being advanced inside the patient. The bending of the tubular member 1 may also allow the catheter to be anchored against tissue after the catheter has been desirably advanced inside the patient. After the catheter has been desirably placed inside the patient, the channel in the flexible line 4 may deliver a substance (e.g., medication such as surfactant) into the channel in the bead, and the substance then exits through a port at the bead. Additionally, or alternatively, the port at the bead may be used to extract substance from inside the patient. For example, suction may be applied at the proximal end of the flexible line 4 to extract substance (e.g., biological sample) through the channel of the bead. The channel in the bead is advantageous because it allows substance to be delivered into the patient, or allows substance to be extracted from the patient, without obstruction of the flow by the bead.

In other embodiments, the tubular member 1 of the catheter of FIG. 6 may have a Y configuration ending in two separate ports. One of ports accommodates the flexible line 4. In particular, the flexible line 4 may exits through one of the ports, which allows for the bending and adjustment of the curvature of the tubular member 1. The other port may be used to deliver substance into the patient, and/or to extract substance from the patient. In such cases, the bead may not include the channel, because the other port can be used for substance delivery.

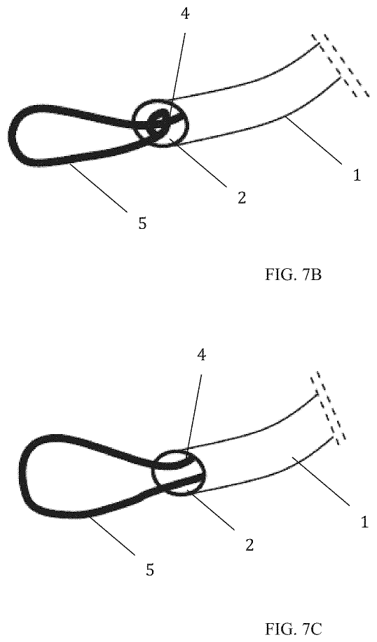

FIG. 7A illustrates a catheter in accordance with other embodiments. The catheter is similar to that described with reference to FIG. 5, except that the flexible line 4 has a solid cross section, and it is not a substance delivery tube.

FIG. 7B illustrates an implementation of the catheter of FIG. 7A, particularly showing a single line 4 exiting the distal end of the tubular member 1 to form a loop (as the anchor 5). The end of the loop forms a small opening for allowing the single line 4 to extend therethrough.

FIG. 7C illustrates another implementation of the catheter of FIG. 7A, particularly showing a single line 4 exiting the distal end of the tubular member 1 to form a loop (as the anchor 5). After forming the loop, the line 4 extends back to the lumen 2 of the tubular member 1.

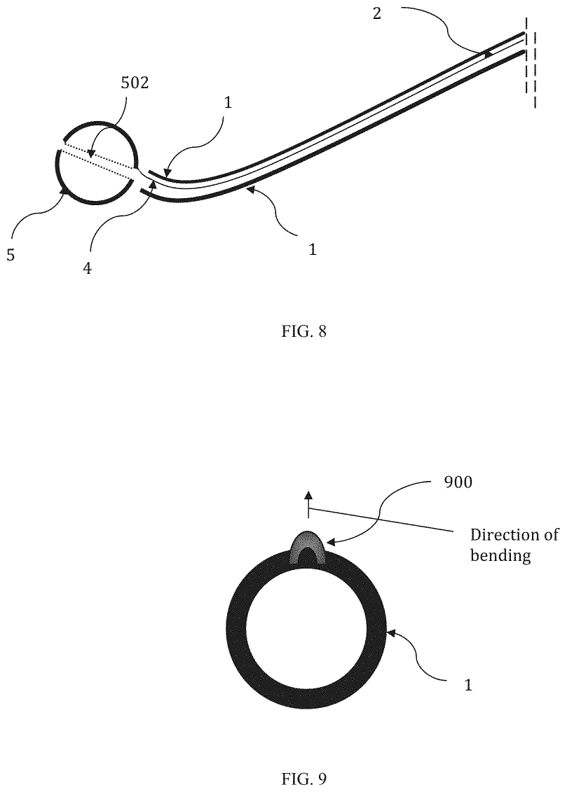

FIG. 8 illustrates a catheter in accordance with other embodiments. The catheter is the same as that described with reference to FIG. 6, except that the flexible line 4 has a solid cross section, and it is not a substance delivery tube.

It should be noted that in the embodiment in which the anchor 5 is a loop, the loop may be advanced distally with respect to the distal end of the tubular member 1. However, in other embodiments, such feature is not required, as the loop does not occlude the fluid delivery port at the distal end of the catheter. Thus, the loop may be maintained attached to the distal end of the tubular member, and the catheter can still deliver substance to within the patient. Similarly, in the embodiment in which the anchor 5 is a bead, the bead may be advanced distally with respect to the distal end of the tubular member 1. However, this feature may not be required if the bead has one or more channels for delivering substance to within the patient. On the other hand, if the bead does not have any channel, then the bead may be advanced distally to open up the fluid delivery port. In other embodiments, if there is a separate fluid delivery port that is different from the tube accommodating the flexible line 4 connecting to the bead, then the bead may or may not be attached to the distal end of the tubular member 1.

As discussed, in some embodiments, the catheter may include a control at its proximal end. In one implementation, two off-the-shelf three-way stopcocks may be coupled in a row to form the control. In such cases, one or two flexible lines 4 may be fed through the stopcocks at their now connected “T” sides. A syringe may be connected to the distal stopcock with the syringe containing the fluid to be instilled. A torsion/wedge may be implemented on the proximal stopcock for applying tension to the flexible line(s) 4, and/or maintaining the achieved tension. Accordingly, such control may be utilized to achieve a desired stiffness and curvature as the procedure proceeds.

Also, in some embodiments that include two flexible lines 4, the two flexible lines 4 may exit the tubular member 1 through a fluid tight split membrane exit port or other opening of similar function of sealing and arresting the flexible line(s). The proximal end of the tubular member 1 may be Y shaped, thus allowing for two separate entry/exit channels that merge towards the distal end (towards or in the patient, away from the operator) of the catheter. One of the arms of the Y ends in the port through which the flexible lines 4 exit. The other arm of the Y provides the entry port for the injection/aspiration of the substance (e.g., surfactant, other preparation, or biological fluid). Traction on either one or both of the flexible lines 4 that exit from a fluid sealed port may used to adjust the size of the distal loop, to put the loop's plane in the direction of the vocal cords, to bend the tubular member 1 in a desired way, or any combination of the foregoing. In some cases, the placement of the loop's plane in the direction of the vocal cords may be achieved with the assistance of the slanted distal tip surface (if it is available) of the tubular member 1.

In some embodiments, the catheter may optionally include a bending (shaping) element configured to pre-bend or bias the tubular member 1 in a certain direction. FIG. 9 illustrates a cross section of a catheter, particular showing the catheter having a bending element 900 coupled to the tubular member 1 of the catheter. The bending element 900 may be a stiffening structure that is secured to an exterior surface or to an interior surface of the tubular member 1. Alternatively, the bending element 900 may be embedded within the wall of the tubular member 1. The bending element 900 may extend at least along a segment (e.g., a distal segment) of the tubular member 1. In the illustrated embodiments, the bending element 900 is slightly shorter compared to the segment of the tubular member 1 to which the bending element is coupled. This has the effect of compressing one side (i.e., the side with the bending element 900) of the tubular member 1, causing the tubular member 1 to pre-bend or to bias towards the side with the bending element 900. In some embodiments, the pre-bending occurs at the distal end of the tubular member 1, and the flexible line(s) 4 may be used to further bend the tubular member 1 in a desired direction. In some cases, the bending element 900 may force the tubular member 1 into a curvature that facilitates its entry into the patient, e.g., into the tracheal.

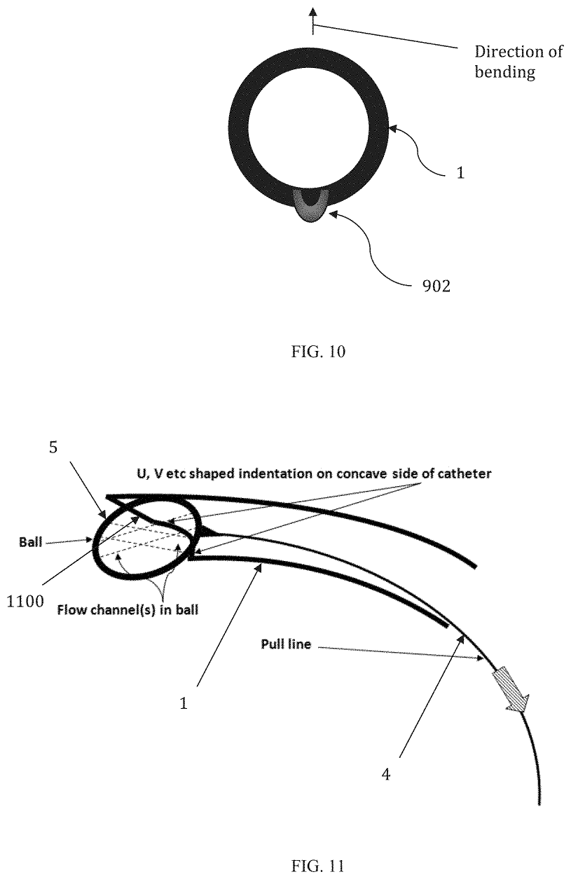

FIG. 10 illustrates a cross section of a catheter, particular showing the catheter having a bending element 902 coupled to the tubular member 1 of the catheter. The bending element 902 may be a stiffening structure that is secured to an exterior surface or to an interior surface of the tubular member 1. Alternatively, the bending element 902 may be embedded within the wall of the tubular member 1. The bending element 902 may extend at least along a segment (e.g., a distal segment) of the tubular member 1. In the illustrated embodiments, the bending element 902 is slightly longer compared to the segment of the tubular member 1 to which the bending element is coupled. This has the effect of tensioning one side (i.e., the side with the bending element 902) of the tubular member 1, causing the tubular member 1 to pre-bend or to bias away from the side with the bending element 902. In some embodiments, the pre-bending occurs at the distal end of the tubular member 1, and the flexible line(s) 4 may be used to further bend the tubular member 1 in a desired direction. In some cases, the bending element 902 may force the tubular member 1 into a curvature that facilitates its entry into the patient, e.g., into the tracheal.

In the above embodiments of FIGS. 9 and 10, the bending (shaping) element provides a bended configuration for the tubular element 1. Accordingly, even without tensioning of the flexible line(s) 4, the tubular element 1 will have a curvature in its “relaxed” state. The relaxed curvature may indicate visually to an operator the direction the catheter will bend when the flexible line 4 is pulled. In other embodiments, the bending element is not required, and the tubular element 1 may not have any curvature when in its relaxed state.

In other embodiments, the shaping element may not apply a compression or tension force on one side of the tubular element 1. Instead, the shaping element may itself has a bent shape, e.g., a curvilinear shape. The shaping element may be stiffer than then tubular member 1. Accordingly, when the shaping element is coupled to the tubular element 1, the shaping element will bend the tubular element 1 and shape it according to the profile of the shaping element. In such cases, the shaping element is not required to be longer or shorter to the segment of the tubular element 1 to which it is coupled.

In further embodiments, the catheter may include both the bending element 900 and the bending element 902 on opposite sides of the tubular member 1.

In other embodiments, instead of using a bending (shaping) element, the tubular member 1 may have a thickened wall on one side compared to other sides. This would provide the tubular member 1 with a desired stiffness, biased-bending direction, and desired curvature.

As discussed, in some embodiments, the catheter may be configured to place the plane of the loop at a certain orientation. For example, the distal end of the tubular member 1 of the catheter may have a shape or profile (e.g., a U or V shape orifice facing a direction of bending of the tubular member 1) that biases the loop to orient in a certain direction when the flexible line 4 is pulled to place the loop in abutment with the distal end of the tubular member 1. FIG. 11 illustrates a distal (first) end of a tubular member of a catheter. As shown in the figure, the distal end of the tubular member 1 has a distal port on a distal tip surface, wherein the distal tip surface 1100 forming an acute angle with respect to a longitudinal axis of the tubular member. In some embodiments, the above features may be implemented to place the plane of the loop/bead to be parallel with the vocal cords when the catheter is inserted into a patient. This allows the distal end of the catheter to be easily slided over the epiglottis into the trachea of the patient. In some cases, the orientation of the distal tip surface 1100 may be achieved by making a slanted cut at the end of the tubular member 1. This creates an elongated slot that defines a plane of the anchor 5. During use, when the anchor 5 is pulled proximally, the elongated slot at the distal tip surface 1100 will guide and rotate the anchor 5 so that it is at a certain orientation with respect to the distal end of the tubular member 1.

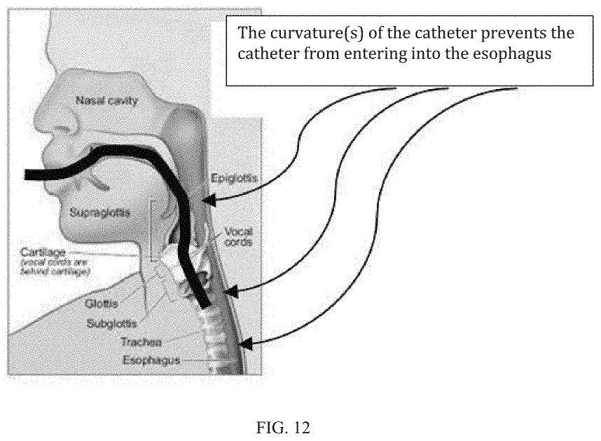

As discussed, in some embodiments, the catheter described herein may be placed into the trachea below the vocal cords for delivering medication, such as surfactants, to a patient. FIG. 12 shows an example of the placement of the catheter in accordance with some embodiments. In the illustrated example, the catheter is inserted into the patient's mouth. As the catheter is advanced, the flexible line(s) 4 may be pulled at the proximal end to bend the distal end of the catheter. The catheter with the curvature is advanced and slided over the epiglottis, and into the trachea and below the vocal cords. The curvature of the catheter is advantageous because it ensures that the catheter is not advanced incorrectly into the esophagus. In some embodiments in which the catheter includes a loop, the plane of the loop may be aligned with the vocal cords and curvature of the tube. For example, the loop may be aligned with the split between the vocal cords. In some cases, the insertion of the catheter may be achieved by visualizing the epiglottis and taking advantage of the curvature of the catheter, and sliding the looped or otherwise blunted tip over the epiglottis into the trachea while avoiding the path into the esophagus. Also, in some embodiments, the tip of the catheter may not need to pass beyond the vocal cords. These normally open under inspiration (breathing in).

The features of the catheter described herein are advantageous because they facilitate the operator's control over the catheter in a challenging small operating field, and they make it easy for such a catheter to be introduced into the trachea and advanced beyond the vocal cords, or into any other part of a human or animal anatomy.

In some embodiments, the catheter described herein may be mass-manufactured. In other embodiments, the catheter described herein may be made ad-hoc at a field (e.g., battle field, jungle, village, etc.) using readily available items, such as fishing line, fiber, thin tubes, cigarette lighter, a match, etc. In one implementation, for emergency purposes, the catheter described herein may be made ad-hoc by inserting a fishing line into a thin tube. One end of the fishing line may be heated using the cigarette lighter or the match to form a melted blob that will function as the anchor 5. If a three-way stopcock is available, it may be attached to the proximal end of the fishing line, and functions as a control for bending the thin tube.

Once the catheter is made on the spot, it may be placed inside the patient, and the fishing line may be used to steer the distal end of the catheter as the catheter is being advanced inside the patient. Once the catheter is desirably placed inside the patient, it may be used to deliver a substance (e.g., medication), and/or it may be used to retrieve an item from the patient. For example, the catheter may be used to obtain biopsy from the patient, provide drainage, retrieve lost and/or foreign bodies from within the patient. By means of non-limiting examples, the catheter may be placed in the ear canal, esophagus, trachea, main stem bronchus, pleura and peritoneal space, vessels (e.g., to remove thrombi, blood clot, plague, etc.), liver, heart, lung, etc.

It should be noted that the applications provided by the catheter described herein are not limited to the examples mentioned above, and that the catheter may be used in other applications. For example, in other embodiments, the catheter may be used as a leading trocar for intubations, including those of a specific bronchus, over which suitable endotracheal or other tubes or catheters can be advanced to their desired location(s). In further embodiments, the catheter may be used as a feeding tube.

Although particular embodiments have been shown and described, it will be understood that they are not intended to limit the claimed inventions, and it will be obvious to those skilled in the art that various changes and modifications may be made without departing from the spirit and scope of the claimed inventions. The specification and drawings are, accordingly, to be regarded in an illustrative rather than restrictive sense. The claimed inventions are intended to cover alternatives, modifications, and equivalents.

What is claimed:

1. A catheter comprising:
a tubular member having a first end, a second end, a body extending between the first end and the second end, and a first lumen in the body;
a first flexible line having at least a segment located in the first lumen of the body; and
a first anchor at an end of the first flexible line, the first anchor having a cross sectional dimension that is larger than a cross sectional dimension of the first lumen;
wherein the first anchor is configured to apply a compression force against an exterior surface at the first end of the tubular member in response to tension applied through the first flexible line.

2. The catheter of claim 1, wherein, in a first operational mode, the first anchor is configured to engage against the first end of the tubular member to allow a bending of the tubular member to occur based on the tension applied through the first flexible line; and
wherein, in a second operational mode, the first anchor is moveable away from the first end of the tubular member in response to a pushing force applied through the first flexible line.

3. The catheter of claim 1, wherein the tubular member comprises a second lumen in the body.

4. The catheter of claim 1, wherein the first anchor comprises a spherical configuration or an ellipsoidal configuration.

5. The catheter of claim 1, wherein the first anchor and the first flexible line are made from different respective materials.

6. The catheter of claim 1, wherein the first anchor and the first flexible line are made from a same material.

7. The catheter of claim 1, wherein the first anchor and the first flexible line have an unity configuration.

8. The catheter of claim 1, wherein the first anchor and the first flexible line are integrally formed together, and wherein the first anchor is a thickened tip of the first flexible line.

9. The catheter of claim 1, wherein the tubular member is configured to form a pig-tail configuration in response to the tension applied through the first flexible line.

10. The catheter of claim 1, wherein the tubular member is configured to form a reversible catheter-retaining hook in response to the tension applied through the first flexible line.

11. The catheter of claim 1, wherein the tubular member has a port at the first end of the tubular member, and wherein the first anchor is configured to close the port.

12. The catheter of claim 11, wherein the first anchor is configured to fluidly seal the port.

13. The catheter of claim 1, wherein the first anchor and/or at least a part of the first flexible line comprises a sampler.

14. The catheter of claim 13, wherein the sampler is configured to obtain a biological sample.

15. The catheter of claim 1, further comprising a second flexible line.

16. The catheter of claim 15, wherein the end of the first flexible line and an end of the second flexible line are configured to couple to the tubular member at different respective longitudinal positions along a longitudinal axis of the tubular member.

17. The catheter of claim 15, wherein the end of the first flexible line and an end of the second flexible line are configured to couple to the tubular member a same longitudinal positions along a longitudinal axis of the tubular member.

18. The catheter of claim 1, wherein the first anchor comprises a loop formed by an elongated element.

19. The catheter of claim 18, wherein the elongated element is an extension of the first flexible line.

20. The catheter of claim 19, wherein a first end of the loop extends to the first flexible line, and a second end of the loop extends to a second flexible line, and wherein a size of the loop is adjustable via manipulation of the first flexible line and/or the second flexible line.

21. The catheter of claim 20, wherein the loop has a bending limit that prevents the loop from being completely pulled inside the tubular member.

22. The catheter of claim 1, wherein the first anchor comprises a sphere or ellipsoid with a fluid delivery channel.

23. The catheter of claim 1, wherein the first flexible line comprises a fluid delivery lumen.

24. The catheter of claim 1, wherein the first end of the tubular member has a distal port on a distal tip surface, the distal tip surface forming an acute angle with respect to a longitudinal axis of the tubular member.

25. The catheter of claim 24, wherein the first end is configured to align a plane of the first anchor with a vocal cord of a patient.

26. The catheter of claim 1, further comprising a shaping element located at the first end of the tubular member, wherein the shaping element is coupled to a wall of the tubular member, and is configured to bend the first end of the tubular member.

27. The catheter of claim 26, wherein the shaping element is configured to apply tension or compression on one side of the tubular member.

28. The catheter of claim 1, further comprising a first port at the second end configured to accommodate the first flexible line.

29. The catheter of claim 28, further comprising a second port at the second end configured to allow fluid to be delivered therethrough.

30. The catheter of claim 29, further comprising a source of treatment fluid coupled to the second port.

31. A medical method, comprising:
inserting a part of a tubular member into a patient, the tubular member having a first end, a second end, a body extending between the first end and the second end, and a first lumen in the body; and
bending the tubular member using a first flexible line and a first anchor at an end of the first flexible line, wherein at least a segment of the first flexible line is located in the first lumen of the body, the first anchor having a cross sectional dimension that is larger than a cross sectional dimension of the first lumen, wherein the first anchor is configured to apply a compression force against an exterior surface at the first end of the tubular member in response to tension applied through the first flexible line.

32. The method of claim 31, further comprising moving the first anchor away from the first end of the tubular member by applying a pushing force through the first flexible line to expose a part of the first flexible line outside the tubular member.

33. The method of claim 31, wherein the tubular member is bent to form a pig-tail configuration in response to the tension applied through the first flexible line.

34. The method of claim 31, wherein the tubular member is bent to form a reversible catheter-retaining hook in response to the tension applied through the first flexible line.

35. The method of claim 31, wherein the tubular member has a port at the first end of the tubular member, and wherein the method further comprises closing the port using the first anchor.

36. The method of claim 31, further comprising collecting a biological sample using a sampler at the first anchor and/or at a part of the first flexible line.

37. The method of claim 31, further comprising delivering a substance to a location inside the patient using the first lumen or another lumen in the tubular member.

38. The method of claim 31, further comprising advancing the first end of the tubular member over an epiglottis of a patient, and advancing the first end of the tubular member into a trachea of the patient.
